(19) United States
(12) Patent Application Publication
(10) Pub. No.: US 2006/0182790 A1
(43) Pub. Date: Aug. 17, 2006

(54) Dermal medicaments application enhancer

(76) Inventor: Flor Mayoral, South Miami, FL (US)

Correspondence Address: Steven Pinkert;Pinkert Law Firm, P.A., Suite 1528, 9130 S. Dadeland Blvd, Miami, FL, 33156, US

(21) Appl. No.: 11/356,015

(22) Filed: Feb. 17, 2006

Related U.S. Application Data
(60) Provisional application No. 60/653,487, filed on Feb. 17, 2005.

(51) Int. Cl. A61F 13/02; A61K 31/198

(52) U.S. Cl. 424/448; 514/561

(57) ABSTRACT

A topical medicament for use in treating tissues comprising: an enhancer for facilitating non-invasive, transdermal delivery and/or enhancing metabolic effect of a therapeutic dosage of LEVULAN® KERASTICK™ (aminolevulinic acid HCl) into a tissue. The present invention provides a topical, transdermal medicament for use in treating tissues comprising an enhancer for facilitating non-invasive, transdermal delivery of a therapeutic dosage of comprising LEVULAN® KERASTICK™ (aminolevulinic acid HCl) into a tissue for example wherein the enhancing agent is comprising L.M.X.4®The enhancer may be selected from the group consisting of lidocaine, benzyl alcohol, carbomer 940, cholesterol, hydrogenated lecithin, polysorbate 80, propylene glycol, trolamine, vitamin E acetate and water or combinations thereof. The present invention, also, provides a pharmaceutical composition useful for treating tissues in humans and animals which comprises a therapeutically effective amount of LEVULAN® KERASTICK™ (aminolevulinic acid HCl) or pharmaceutically acceptable salt thereof in combination with a synergistically effective amount of at least one enhancer or pharmaceutically acceptable carrier wherein said enhancer is selected from the group consisting of lidocaine, benzyl alcohol, carbomer 940, cholesterol, hydrogenated lecithin, polysorbate 80, propylene glycol, trolamine, vitamin E acetate and water. The invention, also, provides a method of treating or preventing a human or animal afflicted by a diseases comprising topically administering a pharmaceutical composition which comprises of a therapeutically effective amount of aminolevulinic acid HCl in combination with an enhancer and/or a pharmaceutically acceptable carrier. The invention, also, provides a method wherein the composition further comprises a synergistically effective amount of an enhancer and/or pharmaceutically acceptable salt thereof wherein said enhancer is selected from the group consisting of lidocaine, benzyl, carbomer 940, cholesterol, hydrogenated lecithin, polysorbate 80, propylene glycol, trolamine, vitamin E acetate, water and combinations thereof.

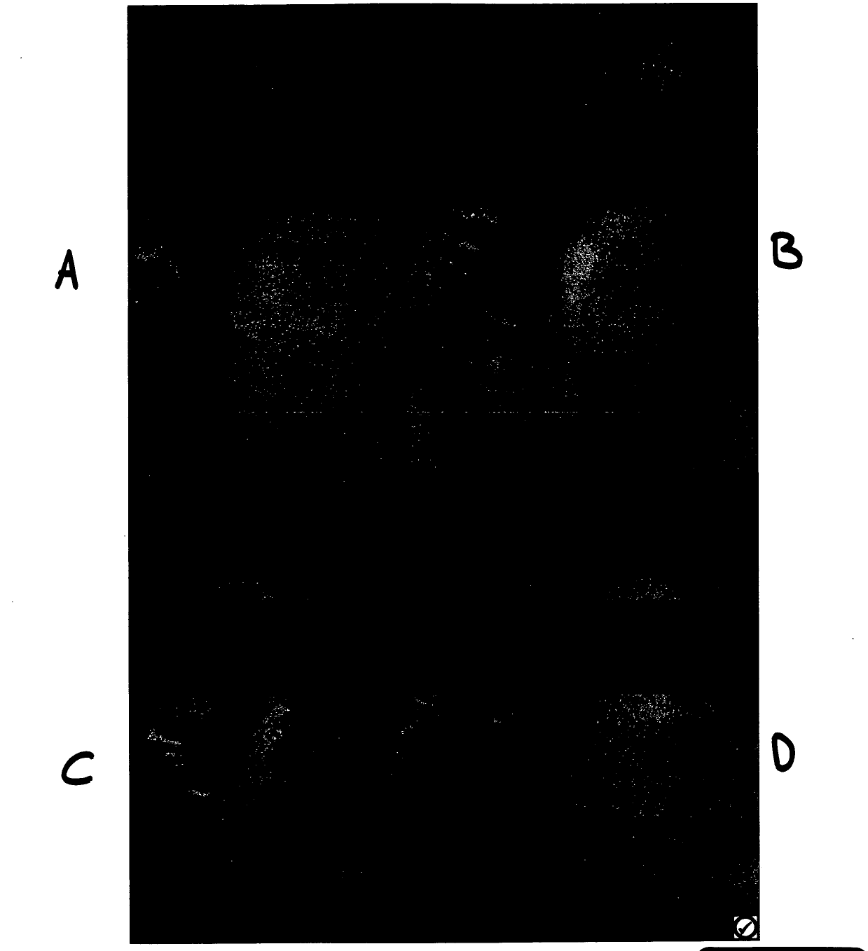

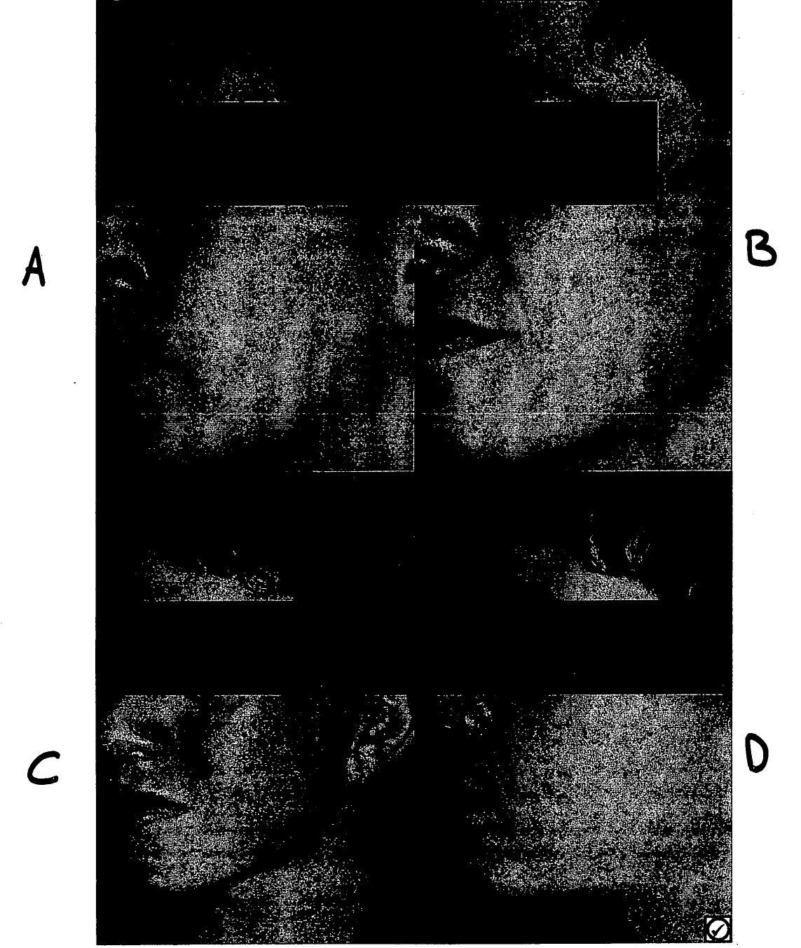

### CROSS-REFERENCE TO RELATED APPLICATIONS

This application claims the benefit of U.S. Provisional Application No. 60/653,487, filed Feb. 17, 2005. The entire disclosure of this prior application is hereby incorporated by reference

### STATEMENT REGARDING FEDERALLY SPONSORED RESEARCH OR DEVELOPMENT

This invention has been created without the sponsorship or funding of any federally sponsored research or development program.

### SEQUENCE LISTING OR PROGRAM

Not available

### FIELD OF THE INVENTION

The present invention relates generally to the field of enhanced activity of topically applied pharmacologically or therapeutically active compounds. In particular, the invention relates to enhancement and control of epidermal, transdermal, and dermal activity of aminolevulinic acid and the pharmaceutical salts thereof. This pharmaceutical is available commercially as the hydrochloride in LEVULAN® KERASTICK™. The enhancer is selected from the group consisting of lidocaine, benzyl alcohol, carbomer 940, cholesterol, hydrogenated lecithin, polysorbate 80, propylene glycol, trolamine, vitamin E acetate, water and combinations thereof. A combination of these ingredients is available commercially and is marketed as L.M.X.4™ which contains benzyl alcohol, carbomer 940, cholesterol, hydrogentated lecithin, polysorbate 80, propylene glycol, trolamine, vitamin E acetate water, and lidocaine.

### BACKGROUND OF THE INVENTION

The topical route of administration of therapeutically active drugs has been used by investigators to deliver the drugs both for the topical effect and the ability to achieve systemic circulation of mammals, including humans by transdermal absorption of the active ingredient.

Various dermal effective pharmacological agents are known which can provide beneficial effects when applied topically to the skin to treat surface or subsurface diseases or for creating skin conditions which protect the skin from external factors. Other pharmacological agents are also known which can provide beneficial effects when absorbed into the systemic circulation. Thus, it is possible to have a systemic effect through topical application of a composition. The topical delivery of systemically effective pharmacological agents can be of significant value in cases where drugs produce gastric problems, are not well absorbed when given orally, or are rapidly metabolized in the liver, e.g. the “first pass” effect. In such cases, the use of topical delivery can give a systemic response at lower dosage than required orally. Topical delivery also avoids the disadvantages present in the intravenous route of administration, which might otherwise be required in order to achieve effective blood levels at reasonable dosage amounts. In addition, dermatological agents can be made more beneficial by enhancing their penetration through the protective layer of the skin or by enhancing the metabolic effect of the dermatological agents. This enhancement of the metabolic effect or penetration is in accordance with the present invention.

The use of penetration or permeation enhancers has been found to be critical to achieve a consistent supply of a therapeutically active ingredient at the site of action during the treatment of skin diseases. For example, as described in U.S. Pat. No. 5,326,566, a composition of such systemically effective pharmacological agents in combination with dibutyl adipate, or a mixture of dibutyl adipate and isopropyl myristate, can greatly enhance the rate of penetration of agents through the skin and can increase the amount absorbed into the systemic circulation. Although a variety of permeation enhancing agents have been used for enhancing the absorption of therapeutic agents into and through the skin, serious problems can arise when permeation enhancers are incompatible with a given drug over a long time period, thereby resulting in drug instability and degradation when the enhancers and the drug are co-formulated into a pharmaceutically acceptable composition for use in warm-blooded mammals, including humans. As a consequence, the practitioner in the art is hampered by an inability to employ certain permeation enhancers for increasing the skin permeation of a drug, if the permeation enhancer and the drug cannot be mixed and stored together without the drug becoming unstable over time and degrading to produce unwanted and potentially harmful byproducts. In addition to the formation of such drug breakdown products, there is also a risk of administering these breakdown products into the circulation of a warm-blooded mammal, including human patients, along with the active drug. Thus, if a drug has demonstrated efficacy in treating a particular affliction of the skin and related tissues, but has a low rate of skin permeation and is unstable for long-term formulation with and storage in permeation enhancing compositions, the utility of such drugs for medical and clinical development and for personal use is greatly diminished, if not abolished.

Accordingly, in view of the foregoing, and because, upon storage, the permeation enhancer degrades the drug in question, or vice versa, one skilled in the art would be led away from using the permeation enhancer with particular drugs with particular permeation enhancers, and vice versa.

Moreover, in certain instances, the permeation enhancer may be incompatible with a drug and/or the composition containing the drug over a long time period, thereby resulting in instability of the permeation enhancer and its degradation when the permeation enhancer and the drug are co-formulated into a pharmaceutically acceptable composition for use in warm blooded mammals, including humans. As a consequence, one skilled in the art is hampered by an inability to employ certain permeation enhancers for increasing skin permeation of a drug, if the permeation enhancer and the drug cannot be mixed and stored together in a pharmaceutically acceptable composition without the permeation enhancer becoming unstable over time and degrading to produce unwanted and potentially harmful products.

Accordingly, the present invention provides an enhanced level of activity for the therapeutic agent when compared with the topical application of the therapeutic agent alone either by enhancing penetration or absorption and or by increasing the metabolic effect of the drug.

Thus, as a result of the present invention, the observed therapeutic effect or activity is greatly enhanced as compared to the therapeutic effect of the drug in the absence of enhancer. The present invention provides a solution to the aforementioned problems in the art by allowing those skilled in the art to utilize a permeation enhancer with a drug, regardless of a penetration rate of the drug, or the incompatibility of the drug with particular permeation enhancers, or vice versa, after being combined therewith.

Grollier et al., U.S. Pat. No. 4,823,985, teaches the use of a dispensing assembly for at least two constituents used in hair coloration and having specified viscosities to enable the common dispensing of the constituents and the production of a composition on a site of application. Grollier et al. describe the preparation of cosmetic products for hair care and, unlike the present invention, are not concerned with the permeation-enhanced, transdermal delivery of a drug having a low permeation rate and an incompatibility with a permeation enhancing agent with which it is combined.

Petersen et al., U.S. Pat. No. 5,156,846, discloses a percutaneous drug delivery system and method which requires pretreating the skin with a skin permeation enhancer, which is an enzyme preparation, and occluding the area of the skin to which the skin permeation enhancing enzyme preparation is applied, removing the skin occlusion means, and applying a drug after rinsing the area. It is disclosed that the skin can again be occluded following application of the drug on the enzyme-pretreated site.

Y. Chang, U.S. Pat. No. 4,956,171, teaches a transdermal drug delivery system having a dual permeation enhancer in which the specific permeation enhancers are sucrose cocoate and methyl laurate. These two enhancers are required for use due to their ability to synergize for penetration enhancement. Unlike the present invention, the disclosure does not relate to a drug and/or permeation enhancer that are unstable during long-term storage with certain permeation enhancers and/or drugs, respectively, which may induce degradation of the drug and/or permeation enhancer, but which can also increase the permeation of the drug when the drug and enhancer are mixed on the skin at the point of use.

German Patent Application No. DE4435805-A1 discloses formation of an enzyme cream at the site of application from an enzyme containing anhydrous ointment base and an aqueous tenside containing oily emulsion (or a mixture of emulsifiers) which are packaged separately. The cream is disclosed to provide enzyme stability and maximum activity at the application site. This patent application deals with an incompatibility between an enzyme and an emulsion vehicle, with the purpose being to maintain enzyme activity at the site of application. This application does not involve or suggest a method and means to increase and enhance the permeation level of a drug which, in the absence of permeation enhancer, has a low level of skin permeation, but which is unstable in the presence of permeation enhancer.

Klein et al., U.S. Pat. No. 4,497,794, discloses a method and composition for the topical treatment of acne, which require the use of the drugs benzoyl peroxide and erythromycin, or derivatives thereof. The patent is not concerned with permeation enhancers and discloses that the two required drug components may be applied to the skin as a mixture or separately applied to the skin; however, unlike the present invention, peroxide, particularly benzoyl peroxide, synergizes with erythromycin. The patentees disclose that erythromycin and benzoyl peroxide, though chemically incompatible, were rendered stable by adding the surfactant, dioctyl sodium sulfosuccinate, to the disclosed benzoyl peroxide/erythromycin gel composition. By contrast, the present invention requires no additives to stabilize the permeation enhancer composition.

Campbell et al., U.S. Pat. No. 4,379,454, discloses a means for delivering to a defined area of skin a co-administered and controlled dosage of a drug and a percutaneous absorption enhancer, with particular regard to a dosage form and method for co-administering estradiol and ethanol percutaneously to treat conditions associated with natural estradiol deficiency.

WO 92/09266, Beecham Group, discloses a two-phase composition for topical drug application in which there are two liquid phases having different lipophilicities. Drug is dissolved in one of the phases such that a supersaturated state with respect to drug must result after mixing the liquid phases.

WO 92/17183, Glaxo, Inc., discloses a sequential dosing medicament and method therefore for topical treatment of fungal infections. The medicament comprises a first composition having an antifungal agent and an anti-inflammatory agent, and a second composition having only an antifingal agent as the active ingredient.

Accordingly, the present invention provides the transdermal delivery of a variety of types of drugs, which otherwise have low permeation rates, at therapeutically effective and significantly increased skin penetration rates, when used and applied as described herein. The present invention further provides the co-delivery and combining of drug having a low rate of skin permeation and a permeation enhancer. Conveniently, the level or extent of penetration of the drug into the skin is advantageously and highly increased at the time of use by mixing or blending the drug and permeation enhancer compositions directly on the skin to produce the desired active composition on the site of application by the user or applier. The present invention also may increase the metabolic activity of the dermatologic agent. The present invention, the enhancer, selected from the group selected from lidocaine, benzyl alcohol, carbomer 940, cholesterol, hydrogenated lecithin, polysorbate 80, propylene glycol, trolamine, vitamin E acetate and water causes an increase in metabolic activity of aminolevulinic acid and the pharmaceutical salts thereof.

### SUMMARY OF THE INVENTION

The present invention provides methods and compositions to enhance the activity of pharmacologically active agents, or drugs, which are delivered topically. This would include increasing the bioavailability of the topical agent. The invention is particularly advantageous for the therapeutic agent aminolevulinic acid and the pharmaceutical salts thereof.

It is an object of the invention to provide an enhanced therapeutic benefit in situ active composition comprising the drug and enhancer for topical delivery. By “enhancer” is meant that the rate or level of pharmacological activity is increased. In accordance with the invention, the therapeutic agent hereafter “drug” and permeation or metabolic enhancer are separately housed and the drug and the permeation or metabolic enhancer are mixed at the site of application on the skin or just prior to application. Also in accordance with the invention, the drug and enhancer are mixed or blended on the site of topical application at the time of simultaneously dispensing each of the component compositions onto the skin, thereby allowing the enhancer to act in combination with the drug at the time of use to significantly increase the expected therapeutic effect of the drug when applied to the skin. In accordance with the invention, the drug and enhancer may be premixed a short time prior to application on the skin, provided that the period of time that the component compositions are premixed results in minimal or virtually no degradation of the drug, thereby allowing a therapeutically effective amount of drug to be transdermally delivered.

It is another object of the invention to provide a method for topical delivery of aminolevulinic acid and the pharmaceutical salts thereof in the form of a composition comprising the drug and a pharmaceutically acceptable vehicle, carrier, or excipient, and forming another composition comprising a enhancing agent, physically separating the compositions, and then co-dispensing and mixing the compositions to form a drug-active composition topically at the point of use. The invention provides an enhanced rate or level of the activity of aminolevulinic acid or its pharmaceutically acceptable salts. For example, it has been observed that when aminolevulinic acid is applied together with or in conjunction with an enhancer within the scope of this invention, the wait time or delay prior to administration or subjection of the patient to activating light, such as Blue Light is greatly reduced. The inventor has observed that the wait period goes from a day or more to a period of hours or even a single hour. Additionally, it has been observed that when aminolevulinic acid is applied together sequentially with the enhancer, after activation with blue light such as BLU-U® there is increased activity of aminolevulinic acid as compared to application of the aminolevulinic acid without enhancer.

It is yet another object of the invention to provide means for housing and delivering at least one drug-containing composition (the drug having a low rate of skin penetration) and at least one permeation or metabolic enhancer composition.

Another object of the invention is to provide a number of different formulations, which are separately housed or compartmentalized prior to application to the skin of the patient in accordance with the invention, two separate formulations are provided: one of a drug, such as aminolevulinic acid or the pharmaceutical salts thereof and a second formulation of the enhancer composition described (selected from the group of lidocaine, benzyl alcohol, carbomer 940, cholesterol, hydrogenated lecithin, polysorbate 80, propylene glycol, trolamine, vitamin E acetate and water). The two formulations are combined and mixed at the site of application at or shortly before the time of application on the skin.

Another object of the invention is to provide three separate formulations: one of drug, preferably in a powder, dry solid or crystalline state, a second formulation of the enhancer, and a third formulation of a vehicle for the combination. The three formulations are combined and mixed at the site of application at or shortly before the time of application on the skin. The formulations are physically separated until application to a body or skin surface and are topically applied, either at the same time, or sequentially within a short time of each other, or may be mixed or blended to form a final active composition, preferably on the skin. In addition, a premixture of the compositions can be made and applied to the skin in accordance with the invention. The invention allows a therapeutically effective amount of drug to be delivered into the skin and systemic circulation and provides significant enhancement of a drug's otherwise low level of skin permeation or metabolic activity by the action of permeation or metabolic enhancer in the active composition at the point of use.

Another object of the invention is a method or a composition of the enhancer to alter photosensitization following application of LEVULAN® Topical Solution, which contains aminolevulinic acid (ALA). Another object of this invention is to alter the metabolic conversion of ALA to PpIX, which accumulates in the skin to which LEVULAN® Topical Solution or aminolevulinic acid, has been applied. In another object of this invention, is to provide a method or a composition which comprises a substance to increase the stability of PpIX. In one object of this invention, the substance is selected from the group consisting of benzyl alcohol, carbomer 940, cholesterol, hydrogentated lecithin, polysorbate 80, propylene glycol, trolamine, vitamin E acetate, water, lidocaine or mixtures thereof. In one object of the invention, the method or the composition comprises one or more these substances. Another object of the invention is to provide a method or composition of the enhancer that increases the metabolic activity or effect of the drug such as aminolevulinic acid.

Further objects and advantages afforded by the invention will be apparent from the detailed description which is set forth herein below.

### DETAILED DESCRIPTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

The information provided below is not admitted to be prior art to the present invention, but is provided solely to assist the understanding of the reader.

The details of one or more embodiments of the invention are set forth in the accompanying drawings and the description below. Other features, objects, and advantages of the invention will be apparent from the description and drawings, and from the claims.

Dermal application offers a series of advantages for a multitude of pharmaceutical substances or other biologically active substances:

the skin is infinitely accessible, no change of medium occurs as in the case of oral application, the operation is easy and convenient, a single dosage suffices, rather than repeated daily doses, positive psychological effects are registered, a continuous long-time therapy is possible, the therapy can be interrupted at any time, a constant plasma level can be guaranteed for a prolonged period, a plasma level which is too high initially, as is the case with intravenous application, is avoided, resulting in negligible secondary action, the danger of an overdosage or underdosage is less, a controlled release of active substances is available, particularly of those with a low therapeutic index.

A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997).

The enhancers known from the literature can be assigned to various chemical classes:

1. Primary and Secondary Alcohols

1.1 Short-chain primary alcohols C2 to C8

1.2 Long-chain primary alcohols C4 to C16

1.3 Secondary alcohols C3 to C5

2. Anionic tensides, such as, for example, Na-dodecylsulphate

3. Saturated and unsaturated fatty acids

4. Azones and derivatives (1-alkyl azacycloheptane-2-on, 1-alkyl azacycloalkanone)

5. Amides such as N,N-diethyl-3-methyl benzamide (DEET), N,N-diethyl-m-toluamide

6. Alkyl-N,N-dialkyl aminoacetate

7. Macrocyclic ketones and lactones

8. Pyrrolidones

9. Esters such as ethyl acetate, isopropyl myristate, glycerine monolaurate, diethyl sebacate, propylene glycol esters of saturated fatty acids

10. Terpenes such as limonene, menthol and cineole

11. Phosphatides

12. Organic acids, such as citric acid, salicylic acid, etc.

13. Cationic tensides or amines.

Bearing in mind both the multitude of interactions taking place within the skin and the varying chemical qualities manifested by the active substances concerned, the penetration enhancing properties of all these so-called enhancers in relation to one active substance can only be predicted with difficulty and in addition to penetration there may be unknown or poorly understood metabolic effects of the enhancers.

From previous experience it can be said that a enhancing substance or a certain mixture only very rarely complies with the characteristics required by several pharmaceutical substances or pharmaceutical substance groups.

From JP-A-61024517 a transdermal therapeutic system for diseases of circulatory organs is known, comprising a plaster with an adhesive layer, a penetration enhancing substance, as well as a beta blocker as active substance. Isopropyl myristate and/or isopropyl lanolate are employed as penetration enhancers. The advantage of this system is said to be a long-lasting administration of the beta-blocking agent, without irritation of the skin occurring. The active substance reaches the blood circulation directly without passing the liver, which means that harmful side effects are avoided. For production of the system, the penetration enhancing substance is incorporated into the adhesive layer, which layer contains the beta blocker as active substance.

From FR-A-21 32 130 cosmetic formulation such as sun creams, facial, body or hand creams are known, in particular, as moisturizers. These are emulsions of the “water in oil” type. To stabilize these emulsions, mixtures of lanolates, such as magnesium lanolate, calcium, lithium, zinc and aluminium lanolate are used. It is the object of the formulation to achieve improved hydration of the epidermis and improved protection thereof. According to example 1, one formulation, for example, contains magnesium lanolate, alcohol of lanoline, isopropyl palmitate, paraffine oil, almond oil, ozokerit, water and parahydroxybenzoate of methyl. The use of polyethyleneglycol ethers of fatty alcohols with longer chains as penetration enhancers in transdermal systems is mentioned, for example, in EP-A-0 189 861, page 10, lines 16 to 24. These penetration enhancers are, for instance, polyoxyethylenealkyl ethers selected from alkyl groups with 4 to 20, preferably 10 to 18, carbon atoms, with the addition of ethylene oxide, such as, for example, polyoxyethylene lauryl ether, polyoxyethylene cetyl ether, polyoxyether stearyl ether and polyoxyethylene oleyl ether. The use of substances in combination with lanoline derivates, however, is neither known from this document, nor does this document render the same obvious.

The present invention provides methods and compositions for enhancing the activity of a topically applied drug or medicament. It is to be understood that the terms drug or medicament denote pharmacologically active compounds or agents (or topically active agents, compounds, or substances) which can confer a therapeutic and/or cosmetic benefit to the user. Such pharmacologically active compounds or agents also may include pharmaceutically acceptable salts or derivatives thereof. It is also to be understood that the terms pharmacologically active compound, drug and medicament are used interchangeably herein. In addition, the terms skin permeation and skin penetration are synonymous as used herein. Here the inventor has determined that the combination of certain topical agents or ingredients results in an enhanced rate or level of the activity for such drugs as aminolevulinic acid or its pharmaceutically acceptable salts. In the case of aminolevulinic acid, for example, it has been observed that when topical agent is applied together with or in conjunction with an enhancer within the scope of this invention, the wait time or delay prior to administration or subjection of the patient to activating light, such as Blue Light is greatly reduced. The inventor has observed that the wait period goes from a day or more to a period of hours or even a single hour. Additionally, it has been observed that when aminolevulinic acid is applied together or sequentially with the enhancer, after activation with blue light such as BLU-U® there is increased activity of aminolevulinic acid as compared to application of the aminolevulinic acid without enhancer.

In addition, the invention provides enhanced therapeutic results for dermal delivery of a topically-applied drug or medicament, which, if normally admixed and stored with particular skin permeation or penetration enhancers prior to use, demonstrates insufficient stability and degrades, thus forming unacceptable drug byproducts, which are potentially deleterious to the user or degrade the effectiveness of the drug. Moreover, the invention also provides enhanced skin therapeutic effect for dermal delivery of a topically-applied drug or medicament which may cause instability and degradation of one or more particular skin permeation enhancers with which the drug is combined or mixed over a long period of time. Accordingly, the invention provides a means for employing virtually any drug with any permeation enhancer to enhance the skin permeation of the drug. The invention is appropriate and especially suitable for the topical application of a drug or drugs which are unstable in the presence of permeation enhancers during long-term storage, and thus, cannot be admixed or combined together for long periods of time prior to use. However the invention is also applicable with drugs that when combined with the enhancer do not degrade over time and thus may be premixed for an extended period of time.

The invention works in conjunction with one or more compositions comprising the drug of interest and a dermatologically-acceptable vehicle for the drug, such that the drug has long-term storage stability in the vehicle and exhibits a first rate of permeation when the drug-containing composition is applied to a body surface or skin area. As a general guide, long-term storage stability refers to the stability of drug when stored for at least about one year, preferably at least two years, and more preferably at least three years, and most preferably at least four years, when stored at room temperature. Provided by the invention are one or more second compositions comprising a skin penetration or permeation or metabolic enhancer for the drug, which, when ordinarily mixed with certain drugs, cause instability of the drug and drug degradation byproducts so that long-term storage stability of the drug in such a mixture is unattainable. The drug-containing composition and the permeation or metabolic enhancer-containing composition are topically applied to an area of the skin, such that the application of the compositions is simultaneous or successive. If the application is successive, one of the compositions must be applied within a short time after the other composition is applied (e.g., within about one to thirty minutes of one another, preferably, within about five to ten minutes of one another, and more preferably, one directly or immediately after the other, e.g., within several seconds).The compositions are mixed, for example, by rubbing (e.g., using finger pressure) at the site of application, thereby forming a blended active composition in situ.

In a preferred mode, one or more drug compositions are first applied to the skin followed by one or more permeation or metabolic enhancer compositions, either immediately or shortly thereafter, i.e., within about ten minutes of one another, preferably within about five minutes of one another, more preferably one right after the other. Alternatively, the drug-containing composition and the permeation or metabolic enhancer-containing composition may be mixed just prior to application, forming a premixture or blended composition, provided that the concentration of the drug is not thereby reduced below 90%, preferably not below 95%, and more preferably, not below 99% of the starting concentration present in the premixed formulation. The premix should be typically applied with regard to the aforementioned concentration requirements. Usually, the premix will be employed within a day, preferably within a few hours, more preferably within about an hour, and most preferably, within minutes after forming the premix. Naturally, the time will vary with storage conditions and overall stability of the drug and permeation enhancer. Those skilled in the art will appreciate that refrigeration will extend the time that the premix can be employed. As a nonlimiting example, room temperature storage conditions may allow the premix to be employed within a day or several days, while refrigeration (e.g., 4-5 DEG C) may extend the time to a month, possibly to about two to three months.

In an alternative mode, when the enhancer does not cause instability of the drug and drug degradation byproducts, the enhancer may be premixed with the drug or the drugs vehicle for an extended period prior to administration to the patient, the most preferred from the time of manufacture.

As described above, when the application of the compositions is in the form of a premix, the time elapsed between the preparation of the premix and its topical application to a body surface or skin area is such that the concentration of the drug in the premix is not less than a predetermined acceptable concentration. For example, the drug concentration in the premix should not be less than 90% of the predetermined acceptable concentration, preferably not less than 95% of the predetermined acceptable concentration, and more preferably not less than 99% of the predetermined acceptable concentration. Upon mixture of the drug composition and the permeation enhancer composition on the skin to form a final active composition comprising drug mixed with permeation or metabolic enhancer, as described hereinabove, a second rate of permeation of the drug is obtained, for example, a rate of at least about 2-fold to 5-fold, preferably at least about 10-fold, more preferably at least about 30-fold, and most preferably at least about 50 to 80-fold, or higher, than the first rate of drug permeation in the absence of the permeation enhancer. Moreover, by physically separating the drug-containing composition and the permeation or metabolic enhancer-containing composition and then mixing the two compositions just at the time of application to produce the active composition on the body surface or skin, or by premixing the two compositions at an appropriate time prior to application on the skin and then applying the premix to the body surface or skin, the production and presence of one or more drug degradation byproducts in the active composition that is delivered to the body surface or skin area (and that could possibly permeate the skin due to the action of the permeation enhancer) are substantially reduced or alleviated.

As described herein, it is to be understood that more than one drug composition and/or permeation or metabolic enhancer composition may be used in the invention, provided that all of the compositions are kept separated during storage and then are combined at the point of use if there is degradation of the drug from premixing for periods of time.

In accordance with the invention, there is provided a method for topical treatment of the human or animal body, comprising applying thereto an effective amount of a pharmaceutical composition comprising a drug and a separately-stored composition comprising an enhancing agent or a permeation or metabolic enhancing agent. The invention is particularly appropriate for a drug that is unstable in the presence of an enhancer or a permeation or metabolic enhancer under the conditions and timeframe of routine storage for pharmaceuticals. The invention further provides a method of obtaining, in a warm blooded mammal, including humans, a desired degree of skin penetration of one or more of a pharmacologically active compound having, on its own, a low rate or level of skin permeation, and capable of having its skin permeation enhanced, but incapable of long-term compatibility or stability with one or more given an enhancer or permeation enhancers.

The proportion of the drug to the permeation enhancer in the active composition may be manipulated as practiced by those in the art by adjusting the concentrations of the components in any suitable manner.

The present invention also encompasses the ability of the skilled practitioner to adjust the degree and amount of permeation into the skin after application of drug and permeation enhancer, for example, by varying the amount of enhancer to produce a predetermined amount for mixing with drug and having maximal thermodynamic activity for permeation. Accordingly, by varying or tailoring the amount or degree of permeation enhancer that is applied, the degree of skin permeation can vary. This aspect of the invention can be achieved by using different means of administration in which the amount of permeation enhancer is adjusted to attain a particular degree of permeation.

Alternatively or concurrently, the present invention also encompasses the ability of the skilled practitioner to adjust the degree and amount of metabolic enhancement after application of drug and enhancer, for example, by varying the amount of enhancer to produce a predetermined amount for mixing with drug and having maximal thermodynamic activity for permeation and or metabolic effect. Accordingly, by varying or tailoring the amount or degree of enhancer that is applied, the degree of skin permeation can vary. This aspect of the invention can be achieved by using different means of administration in which the amount of enhancer is adjusted to attain a particular degree of enhancement of the effect of the drug.

The method in accordance with the invention can be practiced by employing various delivery systems in which the drug-containing compositions and the permeation or metabolic enhancer-containing compositions may be physically separated, compartmentalized, housed, sequestered or stored prior to their actual mixture when applied topically to the skin. As a particular but non-limiting example, if one drug-containing composition and one skin permeation or metabolic enhancer-containing composition are employed, a dual-delivery system is advantageous for use.

In general, devices for housing and dispensing the pharmacologically active composition and the permeation or metabolic enhancer composition of the invention, prior to application and blending of the compositions, may be in the form of collapsible or flexible tubes, rigid tubes, bottles, jars, ampoules, spray dispensers (e.g., aerosol cans), transdermal patches, impregnated pads, syringes, pumps, other dispensers, absorbent applicator, capsules, microcapsules, microparticles, or any other suitable or appropriate presentation known in the art. The dispenser used in the invention will optimally keep the pharmacologically active composition out of physical contact with the permeation or metabolic enhancer composition during storage and until the time of use, or shortly beforehand.

One type of housing and dispensing device is unitary, i.e., comprises a single container which cannot be broken apart, e.g., a single tube or a molded pair of tubes or cylindrical shaped receptacles, whereby the same device and the same dispensing mechanism and aperture are used for application of both the drug-containing composition and the permeation or metabolic enhancer-containing composition. A number of designs for devices suitable for use in the invention are available to those skilled in the art. For example, various dispensers and devices suitable for use, or which may be modified by the skilled practitioner to have the features necessary for separately compartmentalizing and subsequently co-applying or sequentially applying the compositions of the invention, may be found in the disclosures of U.S. Pat. Nos. 4,823,985; 5,156,846; 5,223,262; 4,592,487; 4,379,454; 4,497,794; WO 92/17183; and Spanish Patent P.8803996.

Other dispensing devices useful in the present invention are as described herein below. For example, a single cylindrical jar or container provided with a vertical wall along the diameter line to define two compartments may be used to separate and contain a pharmacologically active composition having a first, low permeation rate (e.g., the first composition) and an incompatible permeation enhancer composition (e.g., the second composition) in which the pharmacologically active composition is unstable, in accordance with the invention. The contents of the compartments are mixed by breaking the vertical dividers just prior to application or use. Also, two separate devices may be used, i.e., one device for each of the first and second compositions.

A single cylindrical jar or container provided with a vertical wall along the diameter line to define three compartments may be used to separate and contain a pharmacologically active composition (drug) in a powder, dry solid or crystalline form in a first compartment, a liquid vehicle to dissolve the drug in a second compartment, and a permeation enhancer composition in a third compartment. The contents of the compartments are mixed by breaking the vertical dividers just prior to application or use.

A single cylindrical jar or container provided with a vertical wall along the diameter line to define two compartments may be used to separate and contain a pharmacologically active composition (drug) in a powder, dry solid or crystalline form in a first compartment, and a liquid vehicle to dissolve the drug combined with the permeation or metabolic enhancer composition in the second compartment. The contents of the compartments are mixed by breaking the vertical dividers just prior to application or use.

A single device may be used for housing and dispensing both compositions, wherein there is a sequential dispensing of the first composition followed immediately or a short time thereafter by the second composition from the same device. The sequential dispensing, i.e., the switching from the first to the second composition, may be manual or automatic. Alternatively, the single dispensing mechanism can be such that the separately housed first and second compositions within in the device are capable of being simultaneously delivered from the same aperture or port, or side-by-side apertures or ports, of the same device at the time of application.

In a manual operation, as but one example, the individual or physician would adjust the device, e.g., by a dial, to dispense the first composition and would then re-adjust the device to dispense the second composition. Alternatively, the device in the manual operation would be preset to dispense the first composition and would then be manually adjusted to dispense the second composition or vice versa. In an automatic operating device, as another example, the device would dispense the first composition which had been pre-filled to a preset amount and only upon its exhaustion would the device then dispense the second composition. In the automatic mode, the individual could be unaware that a different composition was being dispensed after the switch. Alternatively, the device may dispense a preset number of applications of the first composition, i.e., the low permeation rate, pharmacologically-active composition, rather than a preset amount of the first composition. The device would then automatically switch from the first to the second composition; at that point the user would be unable to dispense more and would know to use the second composition at that time. In such a mode, for example, the device would dispense about 7, 14, 28, or 56 times, depending upon whether the first compositions was to be dispensed for 7 or for 14 days and whether dosing was to be 4 times per day, 2 times per day, or 1 time per day. These parameters could be programmed into the device at manufacture or by the pharmacist depending upon the physician's prescription.

For two compositions comprising the invention, wherein one composition comprises a drug in a pharmaceutically acceptable vehicle, carrier or excipient, and the other composition comprises an enhancer or a permeation enhancer, the compositions may be specifically packaged in a dual delivery device, a twin compartment pack, or dermal delivery system ready for topical application by the user or patient. The user or patient would normally apply the two compositions simultaneously on the treatment or skin area and then mix the compositions together in situ via finger pressure and a rubbing motion to create the active treatment composition for topical penetration. The two compositions can also be mixed in the pack or device by breaking a membrane or seal separating the first and second compositions, thereby creating a solution or emulsion in the pack for topical administration or application of the active composition onto the skin. Suitable packs and devices for such purposes are commercially available.

For three compositions comprising the invention, wherein one composition comprises a drug as a powder, dry solid or crystalline form, a second composition contains a pharmaceutically acceptable vehicle, carrier or excipient, and the third composition comprises an enhancer or a permeation or metabolic enhancer, the compositions may be specifically packaged in a dual delivery device, a twin compartment pack, or dermal delivery system ready for topical application by the user or patient. The drug and vehicle can be mixed in the pack or device by breaking a membrane or seal separating them, thereby creating a solution or emulsion in the pack for topical administration or application of the active composition onto the skin.

The user or patient would normally apply the two compositions simultaneously on the treatment or skin area and then mix the compositions together in situ via finger pressure and a rubbing motion to create the active treatment composition for topical penetration with the enhancer. Suitable packs and devices for such purposes are commercially available.

If the device is designed to contain more than two separated compartments, other pharmaceutically acceptable compounds, including different types of drugs and/or permeation enhancing agents, may be stored therein, mixed and dermally administered to an individual as desired or warranted. In this regard, an embodiment of the invention involves more than one drug composition and/or more than one permeation enhancing composition, which can be contained separately, in discrete compartments, containers, reservoirs, and the like, depending upon the nature of the storage and dispensing device, until topically applied on a skin site. Mixing or blending of the various compositions may then occur as described above, at the same time after application, or immediately or a short time following application of the compositions to the skin. In addition, a premixture of the component compositions of the invention, or a subset thereof, can be made immediately or a short time prior to topical application on the skin, and the other component compositions can be admixed and combined with the premixture at the time of topical application.

Thus, in view of the foregoing, it is to be generally understood that a variety of devices and containers for housing and/or storing and applying drug and permeation enhancer and their pharmaceutically acceptable carriers, excipients, or formulations may be used in the invention. The nature of the device should not impact on the scope or operability of the method, provided that the composition comprising drug and the composition comprising permeation or metabolic enhancer are kept separated during storage, and are not allowed to mix or combine until the time of use, immediately before use, or a short time prior to use. The compositions may be premixed, either inside or outside of the device, a short time before use, and then the final active composition is topically applied to the skin. It is understood that if the drug and enhancer are stable they may be premixed for an extended period of time.

In another aspect of the invention, it is envisioned that a plurality of compartments, chambers, reservoirs, containers, bottles, or vials, and the like, depending on the device or delivery means used, can separately contain a number of components comprising the drug composition, and/or the permeation enhancer composition, i.e., multi-component compositions. The separated components are ultimately mixed at or near the time of use to form the composition to be applied. The mixing of the components can occur at the time of topical application and blending on the body or skin surface. Alternatively, the components may be premixed to form the drug composition and the permeation enhancer composition and the two compositions may be applied to the skin and mixed thereon within a defined period of time after the formation of the premixture, thereby forming the therapeutic composition. In this manner, for example, the components of a drug composition may be separated prior to use, and then mixed together to achieve the complete drug composition at the time of use. The drug composition is mixed with the composition comprising the permeation or metabolic enhancer, also at the time of use, to produce the therapeutic drug composition having enhanced skin penetration or metabolic effect.

Drugs useful in the present invention may be in any pharmaceutically acceptable chemical form, such as acid, base, salt, ester, ether, amide, amine, and the like. The drug can be oil-soluble and/or water soluble or soluble in pharmaceutically acceptable solvents, such as alcohols, glycols, and ethers. As but one example of this innovative aspect of the invention, a component of a drug composition comprising a certain acceptable chemical form of the drug, for example, a salt or an acid, is housed in one compartment. A composition comprising a permeation or metabolic enhancer in a formulation for topical application, e.g., gel, cream, ointment, lotion, solution, and the like, is housed in a second compartment. An appropriate excipient vehicle for the drug and permeation enhancer components is housed in a third compartment, the vehicle excipient having particular characteristics, such as pH, ionic strength, buffer capacity, solventability, and the like, which, when mixed with the contents of the first drug compartment and the contents of the second permeation enhancer compartment produces a thermodynamically active composition for the drug and permeation enhancer, such that they have maximum activity to partition and permeate across the skin. All three separately housed compositions are simultaneously or successively applied to the skin and blended or mixed by rubbing on the site of application to form the active composition thereon. The active composition comprises the effective and active chemical form of the drug, which is produced in situ after blending. Preferably, the relative amounts of the solvent, drug, and permeation enhancer present in the final active composition are such that the final composition is saturated with respect to the drug and the permeation enhancer. The pH, solvency, and ionic strength of the final active composition are designed so that maximum thermodynamic activity of the drug and permeation or metabolic enhancer is secured, thus resulting in favorable partitioning of the drug into the skin. In the active composition, the composition comprising the salt or acid form of the drug and the composition comprising the permeation enhancer are mixed so as to enhance the penetration of the active drug into and through the skin by providing an increased penetration rate relative to that possessed by the drug in the absence of the permeation enhancer. This aspect of the invention encompasses the use of a drug in the form of a prodrug in one compartment, which is converted to the active form of the drug after mixing and blending with the appropriate excipient vehicle in a second compartment, as described above, and permeation enhancer on the site of application on the skin.

As a more particular example, the LEVULAN® KERASTICK® composition, may be prepared and placed in one compartment, a vehicle having a pH (e.g., a pH of about 7.9 to 8.5) may be prepared and placed in a second compartment, and a composition comprising an enhancer may be prepared and placed in a third compartment. Following simultaneous application onto the skin, or premixing and then application of the premixture onto the skin a short time after forming the premixture, the three compositions are blended, resulting in the active composition having a high rate of skin penetration and or metabolic enhancing effect. It is preferred that the final mixed active composition be designed such that it provides the maximum thermodynamic activity for the drug and permeation or metabolic enhancer. It will be understood by those in the art that the number of compositions for preparation and use in the invention may be two or more, depending upon the nature of the drug and the formulations desired or needed to comprise the active composition comprising active drug and enhancer. It is also to be understood that one or more drugs and one or more enhancers may be prepared, separately housed, co-applied to the skin, or sequentially applied within a short period of time of each other, if necessary or desired. Of course, when multiple drug and enhancer compositions are employed together, they must be compatible, safe and effective in the active composition that is formed for topical use and skin permeation.

Alternatively, a first composition comprising a drug in a particular vehicle can be prepared and placed in one container and a second composition, which is the solvent for the drug and which also comprises the enhancer, especially an enhancer that causes instability and/or degradation of the drug, can be prepared and placed in a second container. When the first and second compositions are 1) co-applied onto the body or skin and mixed topically on the site; 2) sequentially applied within a short time of one another and mixed on the skin site, or 3) premixed and then applied to the skin a short time after premixing, the drug composition is solubilized in the enhancing solvent composition and the skin penetration of thus-solubilized drug is enhanced over that of the drug in the absence of such solubilization.

It is to be understood that variations of the above-described aspects of the invention are envisioned and contemplated for use, with appropriate modification, if necessary or desired, for storage, delivery, and activity of compositions of drug and an incompatible permeation or metabolic enhancer after topical application.

The compositions of the invention are suitable for any medical, cosmetic, protective, or other treatment of a body surface area.

Examples of dermatologically acceptable vehicles for formulation with a pharmaceutically active agent in the invention include, but are not limited to, any suitable non-toxic or pharmaceutically acceptable topical carrier material or vehicle, such as a solution, suspension, emulsion, lotion, ointment, emollient, salve, unguent, cream, gel, sol, cataplasm, plaster, patch, film, tape or dressing preparation, all of which are well-known to those skilled in the art of topical pharmaceutical formulation.

When a drug and/or permeation or metabolic enhancer fails to give long-term storage stability in the active composition which is designed to yield maximum thermodynamic activity, modifications will have to be made depending upon whether the drug and/or enhancer is oil soluble or water soluble. For example, if the drug is oil soluble, it can be suspended in an aqueous system or it can be solubilized or suspended in a nonaqueous oleaginous system to form a first composition having long-term stability. Similarly, if the enhancer fails to provide long-term stability, it can be isolated away from the drug composition as a second composition. A third composition is then designed so that the drug and the enhancer have maximum thermodynamic activity for drug and enhancer when the final active composition is formed by mixing the three, separate above-described compositions.

In general, the rate of skin permeation (i.e., the maximum thermodynamic activity) is in the order as follows: the skin permeation rate of an optimal solubilized solution (in other words, a saturated solution) is greater than that of a dilute solution. It is necessary to consider the effect of the combination of the solvents in the final active composition on the solubility of the drug whose permeation needs to be enhanced. For acids and bases, the rate of skin permeation is generally higher for unionized forms than for ionized forms. As stated above, it is preferred that the composition containing the permeation enhancer be designed to give the permeation enhancer its maximum thermodynamic activity in the final active composition. For example, permeation enhancers such as menthone and d-limonene perform optimally in alcoholic vehicles, while the skin permeation enhancer sodium lauryl sulfate performs optimally in aqueous compositions containing an optimum level of propylene glycol.

A wide variety of skin penetration or permeation enhancers exist which may be useful in the invention. As an optimal guide, a given permeation enhancer, or combination of enhancers, should provide at least 2-fold to 5-fold, preferably at least 10-fold, more preferably at least 30-fold, and most preferably at least 50 to 80-fold, or higher, enhancement of drug permeation into the skin, compared with the level of permeation in the absence of permeation enhancer. Examples of suitable skin permeation enhancers for use in the invention include, but are not limited to, alcohols (e.g., ethanol, propanol, nonanol); fatty alcohols (e.g., lauryl alcohol); fatty acids (e.g., valeric acid, caproic acid, capric acid); fatty acid esters (e.g., isopropyl myristate, isopropyl n-hexanoate); alkyl esters (e.g., ethyl acetate, butyl acetate); polyols (e.g., propylene glycol, propanedione, hexanetriol); sulfoxides (e.g., dimethylsulfoxide, decylmethylsulfoxide); amides (e.g., urea, dimethylacetamide, pyrrolidone derivatives); surfactants (e.g., sodium lauryl sulfate, cetyltrimethylammonium bromide, polaxamers, spans, tweens, bile salts, lecithin); terpenes (e.g., d-limonene, alpha-terpeneol, 1,8-cineole, menthone); alkanones (e.g., N-heptane, N-nonane); biodegradable skin permeation enhancers (e.g., dodecyl-N,N-dimethylamino acetate, N,N-dimethylamino isopropionate) and water. The permeation enhancers can be formulated as solutions, suspensions, emulsions, gels, creams, lotions, ointments, patches, dressings, liposomes, aerosol sprays, cataplasms, plasters, films, or tape preparations, all of which are well known to those skilled in the art of topical pharmaceutical formulations. It is also appreciated that these enhancers may also exhibit unknown or poorly understood effects on the metabolic rate of the drug they are used in combination with, and in fact what may appear to be a permeation enhancement effect.

The selection of a particular permeation or metabolic enhancer for use in the invention and the determination of the respective concentrations of drug and enhancer can be optimized or changed as necessary or desired by the skilled practitioner by utilizing the assays as described herein.

The compositions of the present invention may also contain other ingredients of the type commonly employed by those skilled in the art of formulating compositions for topical application. These may include, for example, carriers, emollients, surfactants, emulsifying agents, emulsion stabilizing agents, thickening agents, preservatives, anti-oxidants, polymers, chelating agents, fragrances, polymers, adhesives, synthetic membranes, and release liners.

Compared with cream formulations, petrolatum-based ointments generally provide superior skin permeation of pharmacologically active agents contained therein. This is due to the occlusive nature of such ointments. The invention allows the formulation of ointment compositions comprising pharmacological active(s) for mixing with cream compositions comprising permeation enhancer(s) having the appropriate proportions of components in the compositions to produce a cream active composition having an increased rate of skin penetration of the active(s) contained therein. Moreover, petrolatum-based ointments are greasy. In some instances, it may be desired to have less greasy and more cosmetically attractive topical products, such as creams, lotions, gels, and solutions, having skin penetrations of the pharmacological active that are similar to those of a ointment. The production and application of such desired products may be attained by the present invention.

It is to be understood that the term “enhancer” is intended to encompass any biological, chemical or pharmaceutical material, compound or agent which functions as a agent which when used in combination with a topical agent, acts to increase and/or enhance the rate and level of the pharmacologically active compound when applied to the skin. It is further understood that a enhancer or any material serving as an enhancer and/or having the function of an enhancer in accordance with the invention is capable of increasing the rate of penetration or therapeutic activity of the pharmacologically active compound when applied to the skin.

By vehicle is meant a pharmacologically acceptable, inert medium in which an active drug, medicament, or permeation enhancer is administered. Also encompassed by this term are the terms excipient, carrier and placebo, which may be used synonymously herein.

As mentioned above, one of the objects of the present invention is to supply enhancing substances which are tolerated by the skin, are compatible with the active substance involved, do not produce allergies, which are, in addition, easily accessible and economical and, at the same time, possess a therapeutic enhancing effect for one or more active substances.

It has now been discovered, surprisingly, that the application of L.M.X.4® immediately after the application of a composition comprising LEVULAN® KERASTICK® (aminolevulinic acid HCl) has the property of increasing the therapeutic effect and speed of action of the active ingredient of LMX4®. Both of these compositions have been previously known for use in topical application.

LEVULAN® KERASTICK® (aminolevulinic acid HCl) is available as a Topical Solution, 20%, which contains the hydrochloride salt of 5-aminolevulinic acid (ALA), an endogenous 5-carbon aminoketone. This composition is useful in the treatment of non-hyperkeratotic actinic keratoses of the face or scalp. It has also been observed that LEVULAN® is effective in the treatment of Acne Vulgaris (commonly acne). The pathogenesis of Acne is multifactorial including hyper production of sebum from the sebaceous gland in the dermis and proliferation of Propionibacterium Acnes. As it turns out, for reasons unknown LEVULAN® is absorbed rapidly by both sebaceous glands and Propionibacterium Acnes. The effect is reduction in severity of Acne. LEVULAN® is not yet approved for the treatment of Acne, but there are many physicians using it for this purpose. See Hongcharuet al.,(2000) J. Invest. Derm. 115:183-192. Generally at this point LEVULAN® is used for the treatment of Acne when it is at least moderate in severity and the patient is either unwilling, unable or non-responder to conventional antibiotics (oral or topical), isotretinoin, or topical retinoids

Aminolevulinic acid HCl (ALA HCl) is a white to off-white, odorless crystalline solid that is very soluble in water, slightly soluble in methanol and ethanol, and practically insoluble in chloroform, hexane and mineral oil. The chemical name for ALA HCl is 5-amino-4-oxopentanoic acid hydrochloride (MW=167.59).

LEVULAN® KERASTICK® for Topical Solution applicator is a two component system consisting of a plastic tube containing two sealed glass ampoules and an applicator tip. One ampoule contains 1.5 mL of solution vehicle comprising alcohol USP (ethanol content=48% v/v), water, laureth-4, isopropyl alcohol, and polyethylene >glycol. The other ampoule contains 354 mg of ALA HCl as a dry solid. The applicator tube is enclosed in a protective cardboard sleeve and cap. The 20% topical solution is prepared just prior to the time of use by breaking the ampoules and mixing the contents by shaking the LEVULAN® KERASTICK® applicator. The term “ALA HCl” refers to unformulated active ingredient, “LEVULAN® KERASTICK® for Topical Solution” refers to the drug product in its unmixed state, “LEVULAN® KERASTICK® Topical Solution” refers to the mixed drug product (in the applicator tube or after application), and “LEVULAN® KERASTICK® ” refers to the applicator only.

In using LEVULAN® Topical Solution, the solution is typically applied to the skin of a patient and then allowed to rest in contact with the skin for a period of time. The time period is typically of the order of 16 hours. The skin being treated is then washed or cleansed and the surface of the skin is subjected to one or more light treatments. The light or illumination most often used is the BLU-U®°Blue Light, provided by Photodynamic Therapy Illuminator. The inventor has determined that treatment of a patient with LEVULAN® Tropical Solution in combination with, either together or sequencially, a composition comprising a 4% concentration of lidocaine such as the commercially available lidocaine composition L.M.X.4™. This composition also contains benzyl alcohol 1.5% (as a preservative), carbomer 940, cholesterol, hydrogenated lecithin, polysorbate 80, propylene glycol, trolamine, vitamin E acetate and water such as is available in with permits the subsequent treatment if the light or illumination to be carried out after a much shorter waiting period. Rather than the normal 16 hours, the light treatment may be carried out in only a matter of a few hours or even after only 1 hour. This is an unexpected result not previously known in the art.

According to the presumed mechanism of action, photosensitization following application of LEVULAN® Topical Solution occurs through the metabolic conversion of ALA to protoporphyrin IX (PPIX), which accumulates in the skin to which LEVULAN® Topical Solution has been applied. When exposed to light of appropriate wavelength and energy, the accumulated PpIX produces a photodynamic reaction, a cytotoxic process dependent upon the simultaneous presence of light and oxygen. The absorption of light results in an excited state of the porphyrin molecule, and subsequent spin transfer from PpIX to molecular oxygen generates singlet oxygen, which can further react to form superoxide and hydroxyl radicals. Photosensitization of actinic (solar) keratosis lesions using the LEVULAN®, plus illumination with the BLU-U® Blue Light Photodynamic Therapy Illuminator (BLU-U), is the basis for LEVULAN® photodynamic therapy (PDT). Increased metabolic rate or metabolism as used above when related to the presumed mechanism of action described here and the acceleration or increase in production of the metabolite, in the case of LEVULAN®, presumably PpIX or any unknown aspect of the active effect of the drug.

The LEVULAN® KERASTICK® for Topical Solution plus blue light illumination using the BLU-U Blue Light Photodynamic Therapy Illuminator is indicated for the treatment of non-hyperkeratotic actinic keratoses of the face or scalp.

L.M.X.4® contains a 4% concentration of lidocaine. It also contains benzyl alcohol 1.5% (as a preservative), carbomer 940, cholesterol, hydrogenated lecithin, polysorbate 80, propylene glycol, trolamine, vitamin E acetate and water.

The invention also relates to a composition comprising LEVULAN® KERASTICK® (aminolevulinic acid HCl) in a mixture with L.M.X.4® This The invention relates to a composition comprising LEVULAN® KERASTICK® (aminolevulinic acid HCl) in a mixture with L.M.X.4® contains a 4% concentration of lidocaine. In one object of this invention, This invention also relates a composition comprises LEVULAN® KERASTICK® (aminolevulinic acid HCl) and one or more of these substance: benzyl alcohol 1.5% (as a preservative), carbomer 940, cholesterol, hydrogenated lecithin, polysorbate 80, propylene glycol, trolamine, vitamin E acetate and water.

This invention also relates to a composition comprising L.M.X.4™ and a solution vehicle comprising one of more of a compound that is selected from the group consisting of alcohol USP, water, laureth-4, isopropyl alcohol, polyethylene glycol and mixtures thereof.

This invention also relates to a composition comprising L.M.X.4™ and a solution vehicle comprising one of more of a compound that is selected from the group consisting of alcohol USP (ethanol content=48% v/v), water, laureth-4, isopropyl alcohol, polyethylene glycol and mixtures thereof.

In one aspect of the invention, the premixed composition for topical administration comprises Levulan or aminolevulinic acid in an amount of 1% or higher, more preferably 10% or higher and most preferably 20% or higher. In one aspect of the invention, lidocaine is present in the premixed composition for topical administration in an amount of 1-10%, preferably 1-5% or most preferably 1-4%. Similarly, if the components are to be administered separately, the composition comprising lidocaine would include lidocaine in an amount of 1-10%, preferably 1-5% and most preferably 1-5%.

Moreover, in view of the foregoing description of the invention, it will be recognized that any particular and suitable combination of drug and permeation or metabolic enhancer can be readily determined by those skilled in the art. It is also within the skill of those in the art to vary the respective concentrations of drug and permeation enhancer as necessary or required, so that the benefits and advantages of the invention will be recognized.

### EXAMPLES

The examples as set forth herein are meant to exemplify the various aspects of carrying out the invention and are not intended to limit the invention in any way. Unless otherwise specified, it is to be understood that the concentrations of the component ingredients in the compositions of the invention are in %, w/w, based on the total weight of the composition.

### BRIEF DESCRIPTION OF THE DRAWINGS

In describing the invention, reference will at times be made to the accompanying drawings in which:

Exhibit 1. Right (“R”) side of face: Combination of LEVULAN® and L.M.X.4® (Elamax™), followed by exposure to Blue-U light. Note Elamax™ and L.M.X.4® are used interchangeably herein and are trade names for the same or similar products.

Exhibit 2. Left (“L”) side of face: LEVULAN® alone followed by exposure to Blue-U light.

Each Photograph on Exhibits 1 and 2 labeled “A” is pre-treatment for each side of the face. Each Photograph on Exhibits 1 and 2 labeled “B” is a photograph of each side of the face approximately 1 hour and 45 minutes after treatment. Each Photograph on Exhibits 1 and 2 labeled “C” is a photograph of each side of the face approximately 3 days after treatment. Each Photograph on Exhibits 1 and 2 labeled “D” is a photograph of each side of the face approximately 10 days after treatment.

### Example 1

It is, also, possible to reduce the incubation period for LEVULAN® with application of L.M.X.4®. This was observed during the treatment of a patient, J.E., pictured in EXHIBIT 1 and EXHIBIT 2 for type III acne, that was unresponsive to antibiotics. The patient had been off medications for several weeks.

Applicant applied LEVULAN®, 20%, to entire face with the swab of the LEVULAN® KERASTICK® and immediately applied L.M.X.4® to R side of face to anesthetize for smooth beam laser as the laser treatment is painful.

The intent of the experiment was to determine if combined laser and LEVULAN® on R side of face was superior to LEVULAN® alone on L side of face. Since laser was only being applied to one side of the face the anesthetic agent, L.M.X.4®, had only been applied to one side of the face. Waited (incubated) 60 minutes to allow anesthetic to work and permitted LEVULAN® to incubate. Noticed a yellow discoloration on the R side where the anesthetic and LEVULAN® were applied together. The LEVULAN® and the L.M.X.4® had been applied together and thus the yellow chemical reaction had occurred.

When separately applied LEVULAN® and L.M.X.4® dry clear. In the conventional treatment protocol the LEVULAN® is applied a number of hours (typically 16) before the L.M.X.4® is applied and no color reaction is noted.

The L.M.X.4® anesthetic and LEVULAN® were washed from the entire face (R and L) in preparation for laser. Began smooth beam laser treatment on R side of face and laser was not operating properly (misfired). Entire face was placed under BLU-U light to complete the LEVULAN® protocol. BLU-U light exposure for 8 minutes. The light had been started as per standard procedure by the nurse. Following BLU-U exposure observation of patient under ambient light it was observed that the R side of J.E.'s face was much redder (increased erythema) on the R than the L. The attached photographs Exhibit 1 and Exhibit 2 documents the described results. The Exhibit 1-A is a photograph of the right side of patient's face taken pre-treatment and Exhibit 2-A is a photograph of the left side of the patient's face taken pre-treatment. The Exhibit 1-B is a photograph of the right side of patient's face taken approximately 1 hour and 45 minutes after treatment and Exhibit 2-B is a photograph of the left side of the patient's face taken 1 hour and 45 minutes after treatment. One can observe this increased erythema on the R, LEVULAN® and L.M.X.4®, as compared to the L, LEVULAN® alone, in photo Exhibit 1-B and Exhibit 2-B.

Over the next few days the patient was repeatedly observed. It was noted that the reaction on the R side of the face was significantly more intense than the L.

LEVULAN®—Aminolevulinic Acid (ALA) was approved for the topical treatment of Actinic Keratoses (pre-malignant epidermal skin lesions) in October 2000. The chemistry presumably involves ALA becoming metabolized to Protoporphyrin-IX inside cells where it is applied. Protoporphyrin-IX is normally converted to hemoglobin in Red Blood Cells, and is regulated in cells by ferrochetalase. There is differential uptake in pre-malignant cells presumably because these cells have a higher metabolic rate than normal cells. The Protoporphyrin-IX, in the presence of oxygen and light causes cell death, by generating Oxygen radicals, thus eliminating the pre-malignant lesion. Treatment with LEVULAN® comprises an application step, an incubation period where the LEVULAN® is absorbed and converted to Protoporphyrin-IX, and application of light (varying absorption by wavelength most sensitive to 417-432 nm (blue visible spectrum). LEVULAN® is manufactured by DUSA, http://www.dusapharma.com.

LEVULAN® is also effective in the treatment of Acne Vulgaris (commonly acne). The pathogenesis of Acne is multifactorial including hyper production of sebum from the sebaceous gland in the dermis and proliferation of Propionibacterium Acnes. AS it turns out, for reasons unknown LEVULAN® is absorbed rapidly by both sebaceous glands and Propionibacterium Acnes. The effect is reduction in severity of Acne. LEVULAN® is not yet approved for the treatment of Acne, but there are many physicians using it for this purpose. See Hongcharu et al., (200) J. Invest. Derm. 115:183-192 EXHIBIT Generally at this point LEVULAN® is used for the treatment of Acne when it is at least moderate in severity and patient is either unwilling, unable or non-responders to conventional antibiotics (oral or topical), isotretinoin, or topical retinoids.

Inventor, Dr. Flor Mayoral (M.D. licensed in Florida), had previously decided to study the effect of smooth beam laser combined with LEVULAN® to determine if there was an advantage to using both.

Reducing the Incubation Period for LEVULAN® with Application of L.M.X.4®

Treating a patient, J.E., for type III acne, that was unresponsive to antibiotics. The patient had been off medications for several weeks.

LEVULAN®, 20%, was applied to entire face with the swab of the LEVULAN® KERASTICK®. L.M.X.4 was immediately applied to R side of face to anesthetize for smooth beam laser. (laser is painful) Plan was to determine if combined laser and LEVULAN® on R side of face was superior to LEVULAN® alone on L side of face. Patient waited (incubated) 60 minutes to allow anesthetic to work and LEVULAN® to incubate. Doctor noticed a yellow discoloration on the R side where the anesthetic and LEVULAN® were applied together. When applied separately each dries clear, and in the conventional treatment the protocol the LEVULAN® is applied a number of hours (typically 16) before the L.M.X.4® is applied and no color reaction is noted.

After sixty minutes the anesthetic and LEVULAN® were washed from the entire face in preparation for laser. Laser treatment was begun on R side of face but the laser was not operating properly (misfired). It was decided to forgo laser treatment and complete the LEVULAN® treatment so the entire face was placed under BLU-U light to complete the LEVULAN® protocol. Exposure to BLU-U for 8 minutes.

Light was begun per standard protocol by nurse. After the BLU-U light exposure and under ambient light it was observed that the R side of J.E.'s face was much redder (increased erythema) than the L. Dr. Mayoral photographed this at 1:17 pm. that day. See EXHIBIT 1 and 2. EXHIBIT 1-A and 2-A are pretreatment right and left side of face respectively. EXHIBIT 1-B and 2-B are taken just after exposure to the BLU-U light. Note the increased erythema on 1-B, right side of face, LEVULAN® and L.M.X.4®, as compared to 2-B, L side of face, LEVULAN® alone. EXHIBIT 1-B represents the right side of the face which was treated with the LEVULAN® and the enhancer L.M.X.4® whereas EXHIBIT 2-B was LEVULAN® alone.

Over the next few days the patient was repeatedly observed. It was noted that the reaction on the R side of the face was significantly more intense than the L (compare EXHIBITS 1-C and 2-C, taken three days post treatment and EXHIBITS 1-D and 2-D taken ten days post treatment.

Drug Interactions

There have been no prior formal studies of the interaction of LEVULAN® KERASTICK® (aminolevulinic acid HCl) for Topical Solution with any other drugs, and no drug-specific interactions were noted during any of the controlled clinical trials. It is, however, possible that concomitant use of other known photosensitizing agents such as griseofulvin, thiazide diuretics, sulfonylureas, phenothiazines, sulfonamides and tetracyclines might increase the photosensitivity reaction of actinic keratoses treated with the LEVULAN® KERASTICK® for Topical Solution.

The contents of all patents, patent applications, published articles, books, reference manuals and abstracts cited herein are hereby incorporated by reference in their entirety to more fully describe the state of the art to which the invention pertains.

As various changes can be made in the above-described subject matter without departing from the scope and spirit of the invention, it is intended that all subject matter contained in the above description, shown in the accompanying drawings, or defined in the appended claims will be interpreted as descriptive and illustrative, and not in a limiting sense. Many modifications and variations of the present invention are possible in light of the above teachings. It is therefore to be understood that within the scope of the appended claims, the invention may be practiced otherwise than as specifically described.

Reference is made to standard textbooks and other references (e.g., journal articles) that contain definitions and methods and means for carrying out basic techniques, encompassed by the present invention.

### EQUIVALENTS

Those skilled in the art will recognize, or be able to ascertain using no more than routine experimentation, many equivalents to the specific embodiments of the invention described herein. Such equivalents are intended to be encompassed by the claims.

1. A composition for enhancing the epidermal, transdermal, and dermal activity of a therapeutic agent or a pharmaceutical salt thereof wherein the therapeutic agent comprises aminolevulinic acid in combination with one or more enhancer materials.

2. A composition according to claim 1, wherein the therapeutic agent is LEVULAN® KERASTICK®.

3. The composition according to claim 1, wherein the skin permeation enhancer is L.M.X.4®.

4. The composition according to claim 1, wherein the enhancer is selected from the group consisting of lidocaine, benzyl alcohol, carbomer 940, cholesterol, hydrogenated lecithin, polysorbate 80, propylene glycol, trolamine, vitamin E acetate, water, combinations thereof and a natural or synthetic constituent thereof.

5. The composition according to claim 1, wherein the therapeutic agent and enhancer are separately housed and the therapeutic agent and the enhancer are mixed or blended at the site of topical application on the skin or just prior to application.

6. The composition according to claim 5, wherein the therapeutic agent and the enhancer are mixed or blended on the site of topical application at the time of simultaneously dispensing each of the component compositions onto the skin thereby allowing the enhancer to act in combination with the therapeutic agent at the time of use to significantly increase the expected therapeutic effect of the therapeutic agent when applied to the skin.

7. The composition according to claim 6, wherein the therapeutic agent and enhancer may be premixed a short time prior to application on the skin, provided that the period of time that the component compositions are premixed results in minimal or virtually no degradation of the therapeutic agent, thereby allowing a therapeutically effective amount of drug to be transdermally delivered.

8. A method of treating contact dermatitis, rash, housewives' eczema, atopic dermatitis, seborrheic dermatitis, lichen Vidal, prurigo, drug eruption, solar dermatitis, pruritus cutaneous, psoriasis, or erythema comprising topically applying the composition according to claim 1.

9. A method for enhancement and control of epidermal, transdermal, and dermal activity of a therapeutic agent or a pharmaceutical salt thereof wherein the therapeutic agent or pharmaceutical salt thereof comprises aminolevulinic acid and an enhancer wherein the therapeutic agent or combination is applied to the topical surface to be treated.

10. The method according to claim 9 further comprising irradiating at least a portion of the subject with light at a wavelength absorbed by said aminolevulinic acid or by the pharmaceutical salt thereof, wherein said light is provided by a light source; and wherein said irradiation is administered at a relatively low fluence rate that results in the activation of said aminolevulinic acid.

11. The method of 10, wherein said light source is selected from the group consisting of one or a plurality of: laser diodes; light emitting diodes; electroluminescent light sources; incandescent light sources; cold cathode fluorescent light sources; organic polymer light sources; or inorganic light sources.

12. A device for storage and delivery of at least one therapeutic agent, the device comprises two separate formulations that are compartmentalized prior to application to the skin of the patient: wherein the therapeutic agent, is aminolevulinic acid or a pharmaceutical salt thereof and a second formulation is an enhancer that is selected from the group of lidocaine, benzyl alcohol, carbomer 940, cholesterol, hydrogenated lecithin, polysorbate 80, propylene glycol, trolamine, vitamin E acetate, water, combinations thereof and a natural or synthetic constituent thereof.

13. The device according to claim 12 where two separate formulations are combined and mixed at the site of application at or shortly before the time of application on the skin.

14. A device for storage and delivery of at least one therapeutic agent, wherein the device comprises three separate formulations: one of a drug, a second formulation of an enhancer, and a third formulation comprising a vehicle for the drug; wherein the drug comprises aminolevulinic acid or a pharmaceutical acceptable salt thereof.

15. The device according to claim 14 wherein the three formulations are combined and mixed at the site of application at or shortly before the time of application to the skin.

16. A device for storage and delivery of at least one therapeutic agent, wherein the device comprises three separate formulations including a first formulation of a therapeutic agent, a second formulation of the enhancer, and a third formulation of a vehicle for the therapeutic agent; wherein the therapeutic agent is LEVULAN® KERASTICK®.

17. A composition for topically application to the skin of a patient, comprising:
a) at least one first composition comprising the active compound and a pharmacologically acceptable vehicle for the active compound,
b) at least one second composition separated from the first composition comprising an enhancer for the active compound which, when mixed with the active compound, results in enhanced therapeutic or pharmological activity of the active compound.

18. The composition of claim 17, wherein the active compound is aminolevulinic acid HCl.

19. The composition of claim 18 wherein the aminolevulinic acid HCl is in the formulation LEVULAN® KERASTICK®.

20. The composition according to claim 18, wherein the aminolevulinic acid HCl is present in an amount of 0.001 weight % to 80 weight %, based on the weight of the final composition where the first composition and the second composition have been combined.

21. A compartmented device for storage and delivery of a topically applied LEVULAN® KERASTICK® (aminolevulinic acid HCl), comprising:
a first compartment housing at least one first composition comprising aminolevulinic acid HCl. and
a second compartment, physically separated from the first compartment by a rupturable wall which is rupturable by applying pressure to the upper surface and vigorously rubbing the upper surface and which prevents contact between or diffusion of contents of the first and second compartments of the device, the second compartment housing at least one second composition.
